(19) United States
(12) Patent Application Publication
(10) Pub. No.: US 2024/0399105 A1
(43) Pub. Date: Dec. 5, 2024

(54) TESTING DEVICE, TESTING METHOD, AND ELONGATED MEDICAL BODY

(71) Applicant: Terumo Kabushiki Kaisha, Tokyo (JP)

(72) Inventor: Nozomu Watanabe, Kanagawa (JP)

(73) Assignee: Terumo Kabushiki Kaisha, Tokyo (JP)

(21) Appl. No.: 18/669,816

(22) Filed: May 21, 2024

(30) Foreign Application Priority Data
May 31, 2023 (JP) 2023-089459

(51) Int. Cl. A61M 25/00; A61L 29/08

(52) U.S. Cl. CPC A61M 25/0045; A61L 29/085; A61L 2420/02; A61M 2025/0046; A61M 2205/0222; A61M 2205/3327; A61M 2209/02

(57) ABSTRACT

A testing device includes a tube having a lumen, a board provided with a recessed holding groove for holding the tube in a deformed state, and a collecting container placed on the distal side of the tube and used to store liquid flowing in from an injection tool. The holding groove has a groove shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body. The holding groove deforms and holds the tube according to the groove shape when the tube is fitted into the holding groove.

### CROSS-REFERENCES TO RELATED APPLICATIONS

This application claims priority to Japanese Patent Application No. 2023-089459 filed on May 31, 2023, the entire content of which is incorporated herein by reference.

### TECHNOLOGICAL FIELD

The present disclosure generally relates to a testing device for conducting a test of a lubricating layer formed on an elongated medical body which is inserted into and retracted from a body lumen, a testing method using the testing device, and an elongated medical body including a lubricating layer that has the number of particles equal to or less than a predetermined number when measured by the testing method.

### BACKGROUND DISCUSSION

When, for example, a balloon catheter, or a device for diagnosis or treatment, is inserted into a body lumen such as a blood vessel, an introduction catheter such as a guiding catheter is used to guide the device to a target area. In order to reduce frictional resistance to the body lumen, the outer surface of the introduction catheter is provided with a coated lubricating layer formed of a lubricating material such as a hydrophilic polymer in a predetermined area on the outer surface of the introduction catheter.

Many lubricating layers have difficulty in ensuring a sufficient adhesion strength between the layers and the outer surface of a catheter tube and gradually peel off in the form of particles during use. For this reason, lubricating layers are required to have durability for reducing peeling during use so as to meet acceptance criteria outlined in USP <787>, <788>, and the like.

Although, for example, long-duration heat drying can enhance the durability of a lubricating layer, the process increases the manufacturing time and decreases the production efficiency, which may result in deteriorated lubricity in exchange for the enhanced durability.

Japanese Patent Application No. 2021-045525 A discloses a cable having a coat (lubricating layer) enhanced in wiping durability. This disclosed cable includes a sheath and a coat that covers the periphery of the sheath and closely adheres to the sheath. The coat is formed of a rubber composition containing a rubber component and particles, and the surface of the coat has a coefficient of static friction of 0.5 or less and has wipe resistance. When a long fiber nonwoven fabric using cotton linter containing disinfection alcohol (length 50 mm in a wiping direction) is brought into contact with the surface of the coat in such a manner that a shear stress of 2×10⁻³ MPa to 4×10⁻³ MPa is applied to the surface and when the surface of the coat is wiped 20,000 times through 150 mm in the wiping direction at a speed of 80 times/min to 120 times/min, a difference (absolute value) in coefficient of static friction of the coat before and after the test is 0.1 or less.

As described above, the cable disclosed in Japanese Patent Application No. 2021-045525 A is provided with the coat that satisfies a predetermined criterion when measured by a testing method in which the nonwoven fabric having a length of 50 mm in the wiping direction is brought into contact with a part of the surface of the coat and sled through 150 mm at the time of wiping. When the testing device disclosed in Japanese Patent Application No. 2021-045525 A is used to test durability of the entire lubricating layer formed, for example, in an area of about 30 cm from a distal end of a catheter having a length of about 1 m to 2 m, it is required to increase the size of the device so as to conform a total length of the catheter to the device, which is not realistic. In a test using this testing device, the nonwoven fabric is brought into contact with the lubricating layer which is an object of interest. However, it is difficult to uniformly bring the nonwoven fabric into contact with the entire lubricating layer of the relatively long catheter, and it is difficult to obtain an accurate test result.

### SUMMARY

A testing device and a testing method are disclosed that facilitate particle testing on a lubricating layer of a catheter or other long medical elongated bodies and to provide an elongated medical body that includes a lubricating layer regulated by the method and having a reduced number of friction-derived particles while curbing the rise in frictional resistance during insertion into a body lumen and retraction from the body lumen.

The object of the present disclosure is achieved by any one the following aspects (1) to (6).

(1) A testing device for measuring the number of particles generated from a lubricating layer formed on an elongated medical body, the testing device including: a tube having flexibility and provided with a lumen that communicates a distal end and a proximal end of the tube; a board provided with a holding groove having a recessed shape for holding the tube in a deformed state; and a collecting container placed on distal side of the tube and used to store liquid flowing in from an injection tool, wherein the holding groove has a groove shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body, and the holding groove deforms and holds the tube according to the groove shape when the tube is fitted into the holding groove.

(2) A testing method using the testing device according to (1), the testing method involving: preparing the testing device and the elongated medical body provided with the lubricating layer in an area having a predetermined length from the distal end toward the proximal end; setting the distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point; retracting the elongated medical body from the tube and injecting the liquid into the tube in such a manner that collected liquid stored in the collecting container becomes 100 mL in total; and using the collected liquid to measure the number of particles in the liquid and multiplying an obtained value by 10 to convert the obtained value into the number per 100 mL and regarding a converted value as a sum of the particles.

(3) An elongated medical body including: a main body; and a lubricating layer formed on an outer surface of the main body in a predetermined area, the predetermined area having a distal end and a proximal end, wherein the lubricating layer has no more than 5000 particles having a particle size of 10 μm or more and/or no more than 200 particles having a particle size of 25 μm or more when measured with a testing device that includes a tube having flexibility and provided with a lumen communicating a distal end and a proximal end and a collecting container placed on distal side of the tube and used to store liquid flowing in from an injection tool, the tube being deformed and held to have a shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body, by a testing method involving: preparing the elongated medical body provided with the lubricating layer in an area having a predetermined length from the distal end toward the proximal end of the tube; setting a distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point; retracting the elongated medical body from the tube and injecting the liquid into the tube in such a manner that collected liquid stored in the collecting container becomes 100 mL in total; and using the collected liquid to measure the number of particles in the liquid and multiplying an obtained value by 10 to convert the obtained value into the number per 100 mL and regarding a converted value as a sum of the particles.

(4) An elongated medical body including: a main body; and a lubricating layer formed on an outer surface of the main body in a predetermined area, the predetermined having a distal end and a proximal end, wherein the lubricating layer has no more than 5000 particles having a particle size of 10 μm or more and/or no more than 200 particles having a particle size of 25 μm or more when measured with a testing device that includes a tube having flexibility and provided with a lumen communicating a distal end and a proximal end, a board provided with a holding groove for holding the tube in a deformed state, and a collecting container placed on distal side of the tube and used to store liquid flowing in from an injection tool, the holding groove having a groove shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body, and the tube being deformed and held according to the groove shape when fitted into the holding groove, by a testing method involving: preparing the testing device and the elongated medical body provided with the lubricating layer in an area having a predetermined length from the distal end toward the proximal end; setting a distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point; retracting the elongated medical body from the tube and injecting the liquid into the tube in such a manner that collected liquid stored in the collecting container becomes 100 mL in total; and using the collected liquid to measure the number of particles in the liquid and multiplying an obtained value by 10 to convert the obtained value into the number per 100 mL and regarding a converted value as a sum of the particles.

(5) The elongated medical body according to (3) or (4), wherein the lubricating layer is formed by adding a non-lubricating material to a lubricating material, the lubricating material being a hydrophilic material, and the non-lubricating material being any of vinyl chloride resin, urethane resin, and methylene diphenyl 4,4′-diisocyanate.

(6) The elongated medical body according to any one of (3) to (5), wherein the lubricating layer is formed on the outer surface of the main body in an area of 30 cm from the distal end of the elongated medical body, and the main body has an outside diameter of 2.4 mm.

According to the present disclosure, it is possible to provide a testing device and a testing method that facilitate particle testing on a lubricating layer of an elongated medical body. In addition, an elongated medical body provided with a lubricating layer regulated by the testing method of the present disclosure makes it possible to reduce the number of friction-derived particles while curbing the rise in frictional resistance during insertion into a body lumen and retraction from the body lumen.

### BRIEF DESCRIPTION OF THE DRAWINGS

FIG. 1 is a view illustrating a testing device and an elongated medical body according to this embodiment.

FIG. 2 is a view illustrating a configuration of each unit in the testing device according to this embodiment.

FIG. 3 is a flowchart illustrating a testing method according to this embodiment.

FIG. 4 is a view illustrating an operating state when the testing method illustrated in Example is performed.

FIG. 5 is a view illustrating an operating state when the testing method illustrated in Example is performed.

FIG. 6 is a view illustrating an operating state when the testing method illustrated in Example is performed.

FIG. 7 is a table illustrating test results of Examples.

### DETAILED DESCRIPTION

Set forth below with reference to the accompanying drawings is a detailed description of embodiments of a testing device for conducting a test of a lubricating layer formed on an elongated medical body which is inserted into and retracted from a body lumen, a testing method using the testing device, and an elongated medical body including a lubricating layer that has the number of particles equal to or less than a predetermined number when measured by the testing method. Embodiments herein are illustrated to embody the technical idea of the present disclosure and do not limit the present disclosure. Other embodiments, examples, technical operations, and the like that could be conceived by those skilled in the art without departing from the gist of the disclosure are all included in the scope and gist of the disclosure and included in the disclosure disclosed in the claims and the scope of equivalents thereof.

Furthermore, for the purpose of illustration and for ease of comprehension, the scale, aspect ratio, shape, and the like in the drawings attached may be changed from actual ones as appropriate and illustrated schematically. However, it is noteworthy that the drawings are examples and do not limit the interpretation of the present disclosure.

In the following description, note that ordinal numerals such as “first” and “second” will be given but are used for convenience's sake and do not define any order unless otherwise specified. In addition, the terms “proximal end” and “proximal side” refer to the side of an elongated medical body 100 gripped by an operator (the proximal side of the elongated medical body 100 and the side closer to an insertion start point S of a tube 10), and the terms “distal end” and “distal side” refer to the side to be inserted which is opposite to the proximal end of the elongated medical body 100 (the distal side of the elongated medical body 100 and the side closer to an insertion end point E of the tube 10).

### Testing Device

Hereinafter described is a configuration of a testing device 1 according to this embodiment.

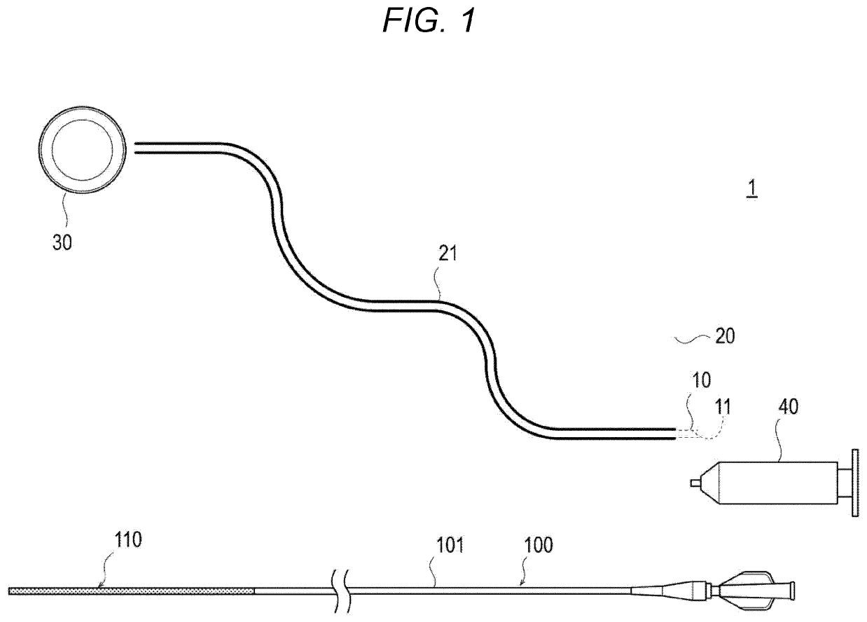

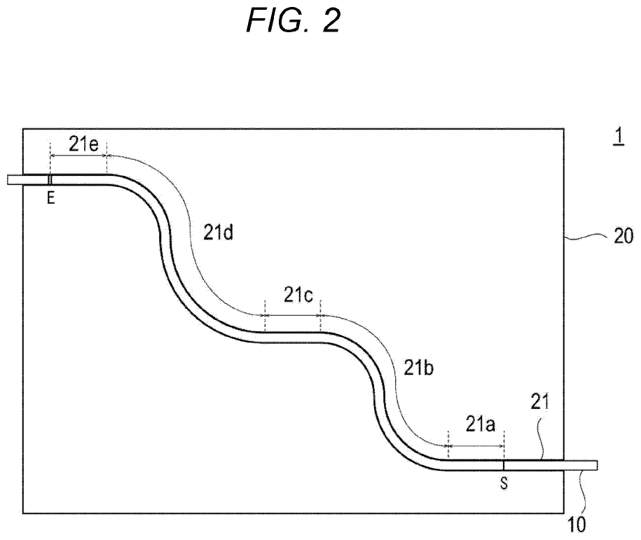

As illustrated in FIG. 1 or 2, the testing device 1 includes the tube 10, a board 20, and a collecting container 30. The testing device 1 is used for inserting and retracting the elongated medical body 100 such as a catheter to measure the number of particles generated from a lubricating layer 110 formed in a predetermined area of the elongated medical body 100.

### Tube

The tube 10 is a hollow tubular member having flexibility and provided with a lumen 11 that communicates a distal end and a proximal end of the tube 10. Liquid such as water or purified water (for example, Distilled water and distilled water) is injected into the lumen 11 of the tube 10 using an injection tool 40 such as a syringe. The inside diameter of the lumen 11 may have a size that allows insertion and retraction of at least the elongated medical body 100 inside the lumen 11 of the tube 10.

The tube 10 may employ a material that has at least some flexibility. With consideration for conformability of a curved portion caused by bending (kinking) when the tube 10 is fitted into a holding groove 21 to form a predetermined curved shape, the tube 10 preferably has a two-layer structure including, for example, an inner layer composed of fluororesin (PVDF-based resin) that has lubricity and an outer layer composed of a urethane-based elastomer that has kink resistance.

### Board

As illustrated in FIG. 2, the board 20 is a table for placing the tube 10 and provided with the holding groove 21 for deforming and holding the tube 10 in a shape suitable for a test.

The holding groove 21 can be, for example, a recessed groove where the tube 10 is fitted and is formed on the upper surface of the board 20. As illustrated in FIG. 2, the holding groove 21 is formed to communicate a right end portion (on the proximal side) and a left end portion (on the distal side).

The holding groove 21 holds the tube 10. The holding groove 21 includes a first straight portion 21a, a first meandering portion 21b adjacent to a distal end of the first straight portion 21a, a second straight portion 21c adjacent to a distal end of the first meandering portion 21b, a second meandering portion 21d adjacent to a distal end of the second straight portion 21c, and a third straight portion 21e adjacent to a distal end of the second meandering portion 21d as seen from an insertion direction of the elongated medical body 100. In this manner, the holding groove 21 has a groove shape in which straight lines (first straight portion 21a, second straight portion 21c, third straight portion 21e) and meandering lines (first meandering portion 21b, second meandering portion 21d) are alternately arranged.

When the tube 10 is fitted into the holding groove 21 and held, the tube 10 deforms and conforms to the groove shape. Accordingly, the tube 10 is deformed by being fitted into the holding groove 21 of the board 20, and like the holding groove 21, and the tube 10 can have a groove shape that follows the first straight portion 21a, the first meandering portion 21b, the second straight portion 21c, the second meandering portion 21d, and the third straight portion 21e of the holding groove 21.

The insertion start point S of the elongated medical body 100 is set in a portion corresponding to the first straight portion 21a of the tube 10, and the insertion end point E of the elongated medical body 100 is set in a portion corresponding to the third straight portion 21e of the tube 10. A test is conducted by inserting the elongated medical body 100 into the tube 10 and by inserting and retracting a portion provided with the lubricating layer 110 in a reciprocating manner between the insertion start point S and the insertion end point E.

The insertion start point S and the insertion end point E in the tube 10 are set at positions that secure at least a length equal to or longer than the area in the elongated medical body 100 provided with the lubricating layer 110. When the elongated medical body 100 is inserted from the insertion start point S to the insertion end point E of the tube 10, the area provided with the lubricating layer 110 is inserted into the tube 10. Therefore, in the elongated medical body 100 having the lubricating layer 110 formed in a range of, for example, 30 cm from the distal end, a length from the insertion start point S to the insertion end point E is preferably at least 30 cm or more.

### Collecting Container

The collecting container (or collection container) 30 is placed on the distal side of the tube 10 and stores (or captures) liquid flowing into the tube 10 from the injection tool 40. The collecting container 30 is capable of storing an amount of liquid necessary for facilitating conversion and obtaining a sum of particles measured by a testing method of the present disclosure. The collecting container 30 is capable, for example, of storing 100 mL according to the testing method.

### Injection Tool

The injection tool 40 is used for injecting liquid into the tube 10. The injection tool 40 is not particularly limited as long as it is capable of injecting liquid into the tube 10. For example, the injection tool 40 can be a syringe that enables an accurate understanding of an amount liquid being injected into the tube 10.

Now specific dimensions of the testing device 1 will be exemplified. The tube 10 may have, for example, an inside diameter of 4 mm, an outside diameter of 6 mm, and a total length of 1 m. The board 20 may have, for example, a length of 230 mm, a width of 380 mm, and a thickness of 19 mm. The holding groove 21 may have, for example, a groove depth of 6.2 mm, a groove width of 6.2 mm, the first straight portion 21a having a length of 80 mm, the second straight portion 21c having a length of 40 mm, the third straight portion 21e having a length of 60 mm, the first meandering portion 21b having a curved portion on the proximal side with a radius of curvature of is 50 mm and a curved portion on the distal side with a radius of curvature is 40 mm, and the second meandering portion 21d having a curved portion on the proximal side with a radius of curvature is 70 mm and a curved portion on the distal side with a radius of curvature is 40 mm.

Note that the tube 10 of the testing device 1 may be formed into a shape that conforms to the holding groove 21 in advance, and then, molded. In this case, when the tube 10 is fitted into the holding groove 21, it is possible to fit the tube 10 smoothly without deformation. The tube 10 herein is fitted into the holding groove 21 and deformed to conform to the shape of the holding groove 21. Alternatively, the tube 10 may be fixed on the board 20 by any fixing tool such as tape in such a manner that the straight lines (first straight portion 21a, second straight portion 21c, third straight portion 21e) and the meandering lines (first meandering portion 21b, second meandering portion 21d) are alternately arranged.

### Testing Method

Hereinafter described is the testing method using the testing device 1.

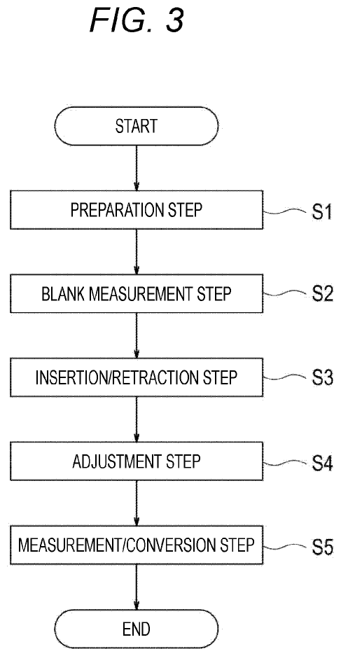

As illustrated in FIG. 3, the testing method according to this embodiment includes a preparation step S1, a blank measurement step S2, an insertion/retraction step S3, an adjustment step S4, and a measurement/conversion step S5. The testing method is not limited to the steps illustrated in FIG. 3, and other steps may be involved. The steps in the testing method are performed in any order as long as they are technically feasible without departing from the gist of the present disclosure.

As illustrated in FIGS. 3 to 6, the testing method is for measuring the number of particles generated from the lubricating layer 110 formed on the elongated medical body 100, or an object to be measured with the testing device 1.

### Preparation Step

The preparation step S1 is to prepare the testing device 1 and the elongated medical body 100 provided with the lubricating layer 110 in an area having a predetermined length from the distal end toward the proximal end. Specifically, in the preparation step S1, preparation of the testing device 1 may involve fitting and setting the tube 10 into the holding groove 21 of the board 20 and placing the collecting container 30 under an end portion of the tube 10 on the distal side (the side closer to the insertion end point E). The preparation step S1 may involve preparing the elongated medical body 100 provided with the lubricating layer 110 in the area having a predetermined length from the distal end toward the proximal end. Note that the preparation step S1 is not necessarily performed immediately before the blank measurement step S2 and may be performed before performing the testing method. In that case, the testing method does not necessarily involve the preparation step S1.

### Blank Measurement Step

The blank measurement step S2 is to record a blank. Specifically, the blank measurement step S2 may involve injecting liquid with the injection tool 40 into the lumen 11 of the tube 10 that has been sufficiently cleaned so as to fill the lumen 11 with the liquid. In the blank measurement step S2, 100 mL of the liquid, for example, can be injected from the proximal side of the tube 10 using the injection tool 40. The blank measurement step S2 may involve measuring 100 mL of the liquid collected in the collecting container 30 using a liquid particle counter under predetermined test conditions. The blank measurement step S2 may involve recording an obtained value as a blank.

The test conditions used in the blank measurement step S2 are as follows.
Amount to be collected: 10 mL per measurement
Frequency of collection: 4 times
Calculation method: discard the first collected liquid and use data of the liquid collected in the remaining three measurements to obtain the average value
Unit: particles/10 mL.

### Insertion/Retraction Step

The insertion/retraction step S3 is to insert and retract the tube 10 a predetermined number of times using the elongated medical body 100 which is a device of interest. Specifically, the insertion/retraction step S3 may involve setting (placing) the distal end of the elongated medical body 100 at the insertion start point S of the tube 10 and repeating insertion and retraction 5 times by inserting the elongated medical body 100 from the insertion start point S to the insertion end point E of the tube 10 in such a manner that the portion of the elongated medical body 100 provided with the lubricating layer 110 is completely inserted into the tube 10 and by subsequently retracting the distal end of the elongated medical body 100 to the insertion start point S.

### Adjustment Step

The adjustment step S4 is to adjust the total amount of liquid collected (collected liquid) in the collecting container 30 to a predetermined amount. Specifically, the adjustment step S4 may involve removing the elongated medical body 100 from the tube 10 and injecting the liquid into the tube 10 in such a manner that the liquid (collected liquid) stored in the collecting container 30 becomes 100 mL in total.

### Measurement/Conversion Step

The measurement/conversion step S5 is to measure the number of particles in the collected liquid and converting an obtained value to determine a sum of particles. Specifically, the measurement/conversion step S5 may involve setting 100 mL of the collected liquid adjusted in the adjustment step S4 to the particle counter and converting an obtained value into the number per 100 mL under test conditions similar to those used in the blank measurement step S2. The measurement/conversion step S5 may involve recording a value obtained by subtracting the value of the blank measured in the blank measurement step S2 as a sum of particles of the elongated medical body 100 which is the object of interest.

The particle counter used in the blank measurement step S2 and the measurement/conversion step S5 is not limited as long as it is a light-shielding liquid particle counter, and the testing method may employ, for example, HIAC 9703 available from Beckman Coulter, Inc.

### Elongated Medical Body

The elongated medical body 100 is an instrument in which the lubricating layer 110 having no more than a predetermined number of particles measured by the testing method is formed on the outer surface of a main body 101 in a predetermined area from the distal end toward the proximal end. Specifically, the elongated medical body 100 can preferably employ the lubricating layer 110 including no more than 5000 particles having a particle size, for example, of 10 μm or more and/or no more than 200 particles having a particle size of 25 μm or more. In other words, as the lubricating layer 110, it is preferable to employ a layer in which the number of particles having a particle size of 10 μm or more and the number of particles having a particle size of 25 μm or more per unit length and per unit area with respect to the surface area of the elongated medical body 100 (pi×outside diameter (2.4 mm)×length (300 mm)=2,261 mm²) are respectively 2 (2.21) particles/mm² and 0.1 (0.088) particles/mm² or less per device.

The lubricating layer 110 is formed of a material having coating strength (peel resistance after formation) that satisfies the aforementioned number of particles. The lubricating layer 110 is formed by adding a non-lubricating material to a lubricating material.

The lubricating material is hydrophilic. A specific example of the hydrophilic material includes a block copolymer of glycidyl methacrylate-dimethylacrylamide.

In particular, the lubricating layer 110 preferably includes acrylic resin as a lubricating resin. The acrylic resin preferably contains a constitutional unit derived from a reactive monomer. Introducing such a constitutional unit derived from a reactive monomer enables crosslinking or polymerization of polymers via an epoxy group, or the reactive monomer, thereby forming a network structure.

The reactive monomer is preferably an ester of (meth)acrylic acid having an epoxy group. In particular, the reactive monomer preferably contains at least one selected from the group consisting of glycidyl acrylate, glycidyl methacrylate (GMA), 3,4-epoxycyclohexyl methyl acrylate, 3,4-epoxycyclohexyl methyl methacrylate, and 3-methyl glycidyl methacrylate because it is easy to control crosslinking or polymerization of polymers. More preferably, the reactive monomer is glycidyl (meth)acrylate, and still more preferably, the reactive monomer is glycidyl methacrylate (GMA). Herein, “(meth)acrylate” represents both acrylate and methacrylate. One type of the reactive monomer may be used independently or two or more types may be used in combination.

In addition, the acrylic resin preferably includes a constitutional unit (A) derived from a reactive monomer having an epoxy group and a constitutional unit (B) derived from a hydrophilic monomer. More preferably, the acrylic resin consists of the constitutional unit (A) derived from a reactive monomer having an epoxy group and the constitutional unit (B) derived from a hydrophilic monomer.

Examples of the hydrophilic monomer include acrylamide and derivatives of acrylamide, vinylpyrrolidone, acrylic acid and methacrylic acid and derivatives vinylpyrrolidone, acrylic acid and methacrylic, polyethylene glycol acrylate and derivatives of polyethylene glycol acrylate, monomers having a sugar or a phospholipid in a side chain, and water-soluble monomers such as maleic anhydride, but the hydrophilic monomer is preferably N,N-dimethylacrylamide (DMAA). One type of the hydrophilic monomer may be used independently or two or more types may be used in combination.

A method for manufacturing the acrylic resin is not particularly limited. For example, a block copolymer having a hydrophilic moiety and a reactive moiety is produced by a method disclosed in Japanese Patent Application Publication No. 9-131396 A.

The non-lubricating material of the lubricating layer 110 refers to a non-lubricating resin or a non-lubricating compound that does not have lubricity by itself. Preferable examples of the non-lubricating resin include vinyl chloride resin (PVC) and urethane resin, and a preferable example of the non-lubricating compound includes methylene diphenyl 4,4′-diisocyanate (MDI).

Examples of the urethane resin include polyester-based urethane resin, polyether-based urethane resin, and polycarbonate-based urethane resin. A urethane elastomer is also applicable, and it is preferable to employ a commercially available urethane elastomer having a polyether segment such as Pellethane 2363 available from Lubrizol and Elastollan available from BASF.

The non-lubricating resin and/or non-lubricating compound of the lubricating layer 110 is preferably insoluble in water. This configuration enhances the durability. The expression “insoluble in water” represents that a substance is insoluble (or hardly soluble) in water at room temperature (23° C.) and normal pressure (1 atmospheric pressure). For example, a substance is referred to as insoluble if less than 1 g of the substance dissolves in 100 mL of water at room temperature and normal pressure but not limited thereto.

A mass ratio of the non-lubricating resin added to the lubricating resin preferably ranges from 1/1000 to 1.

A solvent for dissolving or dispersing the non-lubricating resin and the lubricating resin is not particularly limited as long as it dissolves (or disperses) the non-lubricating resin and the lubricating resin according to the present disclosure. Specific examples of the solvent include, but are not particularly limited to, water, alcohols such as methanol, ethanol, isopropanol, and ethylene glycol, ketones such as acetone, methyl ethyl ketone, and cyclohexanone, esters such as ethyl acetate, halides such as chloroform, olefins such as hexane, ethers such as tetrahydrofuran (THF) and butyl ether, aromatics such as benzene and toluene, amides such as N,N-dimethylformamide (DMF), and sulfoxides such as dimethyl sulfoxide (DMSO). These solvents may be used independently or two or more types may be used in combination. Among the examples, N,N-dimethylformamide (DMF) or tetrahydrofuran (THF) is preferable.

The lubricating layer 110 can be formed by coating or immersing into a solution obtained by adding the hydrophilic material or the like, and then, heating for a predetermined time (for example, for about 1 hour to 6 hours, preferably about 1 hour to 3 hours). The heating temperature can be, for example, 90° C. or more and 150° C. or less (90° C. to 150° C.), preferably 100° C. or more and 140° C. or less (100° C. to 140° C.), and more preferably 100° C. or more and 130° C. or less (100° C. to 130° C.).

Since the lubricating layer 110 of the elongated medical body 100 is formed of the aforementioned material, it is possible to reduce the heating time during formation. Therefore, the lubricating layer 110 can suppress the progress of excessive crosslinking or polymerization. For this reason, the lubricating layer 110 has a low crosslinking density and causes little damage to a hydrophilic group, which diminishes the restriction on movement. Accordingly, the lubricating layer 110 has high durability and maintains surface lubricity even when subjected to friction. In other words, the lubricating layer 110 has a small frictional resistance or does not increase in frictional resistance and makes it possible to reduce the number of particles generated.

The elongated medical body 100 may be any medical instrument as long as it is used during therapy or treatment by being inserted into and retracted from a body lumen. Specific examples of the elongated medical body 100 can include guidewires and various kinds of catheters such as guiding catheter, angiographic catheter, microcatheter, guidewire support catheter, balloon catheter, stent delivery catheter, diagnostic imaging catheter, atherectomy catheter, introducer sheath, and dilator.

As described above, the testing device 1 according to this embodiment is for measuring the number of particles generated from the lubricating layer 110 formed in the elongated medical body 100 and includes the tube 10 having flexibility and provided with the lumen 11 communicating the distal end and the proximal end, the board 20 provided with the recessed holding groove 21 for holding the tube 10 in a deformed state, and the collecting container 30 placed on the distal side of the tube 10 and used to store liquid flowing in from the injection tool 40. The holding groove 21 has a groove shape including the first straight portion 21a, the first meandering portion 21b adjacent to the distal end of the first straight portion 21a, and the second straight portion 21c adjacent to the distal end of the first meandering portion 21b, the second meandering portion 21d adjacent to the distal end of the second straight portion 21c, and the third straight portion 21e adjacent to the distal end of the second meandering portion 21d as seen from the insertion direction of the elongated medical body 100. The holding groove 21 deforms and holds the tube 10 according to the groove shape when the tube 10 is fitted into the holding groove 21.

The testing method according to this embodiment uses the testing device 1 and involves: preparing the testing device 1 and the elongated medical body 100 provided with the lubricating layer 110 in the area having a predetermined length from the distal end toward the proximal end (preparation step S1); setting the distal end of the elongated medical body 100 at the insertion start point S of the tube 10 and repeating insertion and retraction 5 times by inserting the distal end of the elongated medical body 100 from the insertion start point S to the insertion end point E of the tube 10 in such a manner that the portion of the elongated medical body 100 provided with the lubricating layer 110 is completely inserted and by subsequently retracting the distal end of the elongated medical body 100 to the insertion start point S (insertion/retraction step S3); retracting the elongated medical body 100 from the tube 10 and injecting liquid into the tube 10 in such a manner that collected liquid stored in the collecting container becomes 100 mL in total 30 (adjustment step S4); and using the collected liquid to measure the number of particles in the liquid and multiplying an obtained value by 10 to convert the obtained value into the number per 100 mL and regarding a converted value as a sum of the particles (measurement/conversion step S5).

The elongated medical body 100 according to this embodiment includes the main body 101 and the lubricating layer 110 formed on the outer surface of the main body 101 in a predetermined area from the distal end toward the proximal end, and the lubricating layer 110 has no more than 5000 particles having a particle size of 10 μm or more and/or no more than 200 particles having a particle size of 25 μm or more according to the testing method.

With such a configuration, it is possible to provide a testing device and a testing method capable of facilitating particle testing on the lubricating layer 110 of the elongated medical body 100. In addition, the elongated medical body 100 provided with the lubricating layer 110 regulated by the testing method of the present disclosure makes it possible to reduce the number of friction-derived particles while curbing the rise in frictional resistance during insertion into a body lumen and retraction from the body lumen.

### EXAMPLES

The effects of the present disclosure will be described with reference to the following Examples and Comparative Examples. Note that the technical scope of the present disclosure is not limited to the following Examples.

### 1. Specification of Samples

Samples for Examples (Example 1 to Example 3) and samples for Comparative Examples (Comparative Example 1 and Comparative Example 2) were prepared according to the following steps.

A block copolymer, or a lubricating material that constitutes a lubricating layer, was produced by a synthesis reaction indicated by the following Chemical Formula 1.

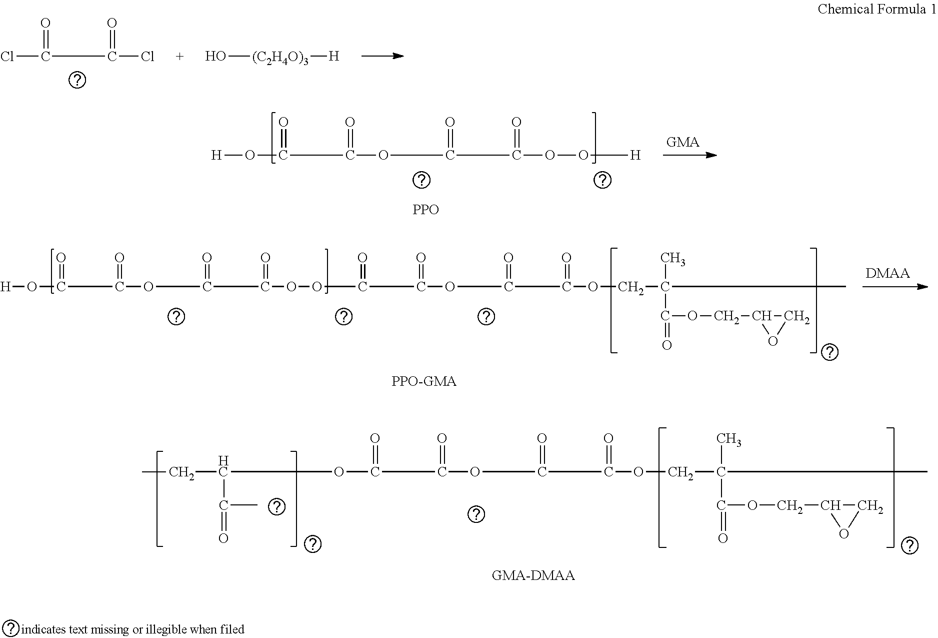

After 29.7 g of triethylene glycol was added dropwise to 72.3 g of adipic acid dichloride at 50° C., hydrochloric acid was removed at 50° C. for 3 hours under reduced pressure to obtain an oligoester. Next, 4.5 g of methyl ethyl ketone was added to 22.5 g of the obtained oligoester, and the mixture was added dropwise to a solution containing 5 g of sodium hydroxide, 6.93 g of 31% hydrogen peroxide, 0.44 g of dioctyl phosphate as a surfactant, and 120 g of water and reacted at −5° C. for 20 minutes. The resulting product was repeatedly washed with water and methanol, and then, dried to obtain a poly peroxide (PPO) having a plurality of peroxide groups in a molecule.

Next, 0.5 g of this PPO, 9.5 g of glycidyl methacrylate (GMA), and also 30 g of benzene as a solvent were polymerized while being stirred at 80° C. for 2 hours under reduced pressure. The reactant obtained after the polymerization was reprecipitated with diethyl ether to obtain polyglycidyl methacrylate having a plurality of peroxide groups in a molecule (PPO-GMA).

Subsequently, 1.0 g of the obtained PPO-GMA (corresponding to 7 mmol of GMA) was charged into 9.0 g of N,N-dimethylacrylamide (DMAA) and 90 g of dimethyl sulfoxide as a solvent, and the mixture was reacted at 80° C. for 18 hours. The reactant obtained after the reaction was reprecipitated with hexane and collected, thereby obtaining a block copolymer (1) (constitutional unit (A): constitutional unit (B)=GMA DMAA=1:14 (molar ratio)). The weight average molecular weight (Mw) of the block copolymer measured by gel permeation chromatography (GPC, polystyrene equivalent) was about 1.5 million.

The obtained block copolymer was dissolved in N,N-dimethylformamide (DMF) to prepare a coating liquid free of non-lubricating resin (coating liquid 1). In addition, a coating liquid to which a non-lubricating resin was added (coating liquid 2) was prepared by dissolving the obtained block copolymer and polyvinyl chloride resin (available from FUJIFILM Wako Pure Chemical Corporation) in N,N-dimethylformamide (DMF) in such a manner that the mass ratio (non-lubricating resin/lubricating resin)=1/45 and the non-lubricating resin in the coating liquid became 0.1 mass %. Furthermore, a coating liquid to which a non-lubricating resin was added (coating liquid 3) was prepared by dissolving the obtained block copolymer and polyvinyl chloride resin (available from FUJIFILM Wako Pure Chemical Corporation) in N,N-dimethylformamide (DMF) in such a manner that the mass ratio (non-lubricating resin/lubricating resin)=1/45 and the non-lubricating resin in the coating liquid became 0.5 mass %. Still further, a coating liquid to which a non-lubricating resin was added (coating liquid 4) was prepared by dissolving the obtained block copolymer and methylenediphenyl 4-4′-diisocyanate (MDI) in N,N-dimethylformamide (DMF) in such a manner that the mass ratio (non-lubricating resin/lubricating resin)=1/45 and a concentration of the coating liquid became 5.3 mass % and MDI in the coating liquid became 0.1 mass %. As described above, the coating liquid 1 was composed of the lubricating resin, and the coating liquids 2 to 4 included the lubricating resin to which the non-lubricating resin was added.

A guiding catheter having an outside diameter of 2.4 mm, an inside diameter of 2.2 mm, and a total length of 150 cm (R2P® SlenGuide®: available from Terumo Corporation) was used as the elongated medical body 100, an area of 30 cm from a distal end of a main body of the catheter was immersed in any one of the coating liquids prepared above, thereby drying the catheter at room temperature (25° C.) for 1 hour to form a coating film. Furthermore, the elongated medical body 100 was subjected to heat drying in an oven at 130° C. over a predetermined time, and then, cooled to room temperature. In this manner, samples of the elongated medical body 100 provided with the lubricating layer 110 were prepared.

The type of coating liquid used in each sample and the heating time were as follows.
Sample 1 (Example 1): coating liquid 2, 1.5 hours
Sample 2 (Example 2): coating liquid 3, 1.5 hours
Sample 3 (Example 3): coating liquid 4, 1 hour
Sample 4 (Comparative Example 1): coating liquid 1, 1.5 hours
Sample 5 (Comparative Example 2): coating liquid 1, 12 hours.

### 2. Preparation of Testing Device

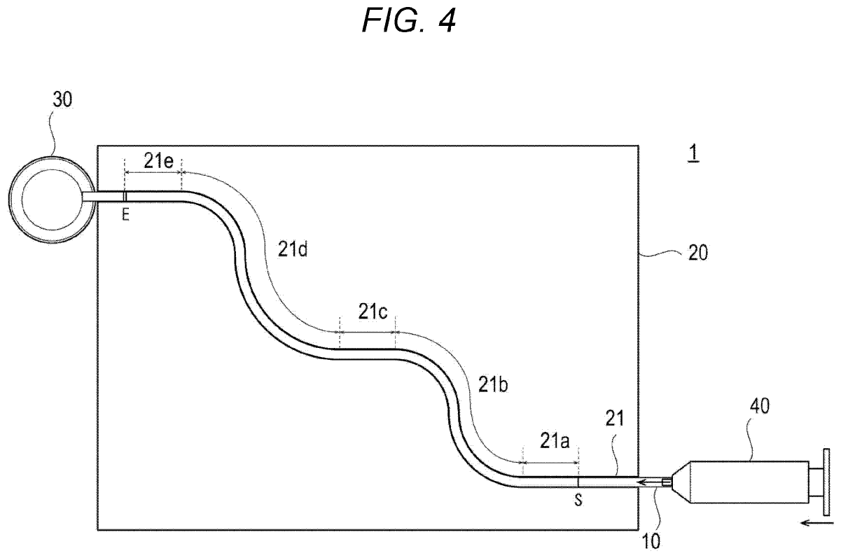

As illustrated in FIG. 4, in the testing device 1, the upper surface of the board 20 was provided with the holding groove 21 where straight lines (first straight portion 21a, second straight portion 21c, third straight portion 21e) and meandering lines (first meandering portion 21b, second meandering portion 21d) are arranged alternately, and the tube 10 was fitted into the holding groove 21. With regard to the tube 10, employed was a hose having an inside diameter of 4 mm, an outside diameter of 6 mm, and a total length of 1 m (E-PD-4 flexible fluorine hose (tube type), available from HAKKO CORPORATION). The board 20 had a length of 230 mm, a width of 380 mm, and a thickness of 19 mm. The holding groove 21 had a groove depth of 6.2 mm, a groove width of 6.2 mm, the first straight portion 21a having a length of 80 mm, the second straight portion 21c having a length of 40 mm, the third straight portion 21e having a length of 60 mm, the first meandering portion 21b having a curved portion on the proximal side with a radius of curvature is 50 mm and a curved portion on the distal side with a radius of curvature is 40 mm, and the second meandering portion 21d having a curved portion on the proximal side with a radius of curvature is 70 mm and a curved portion on the distal side with a radius of curvature is 40 mm.

### 3. Testing Method

The testing method was performed in the following manner.

First, as illustrated in FIG. 4, the tube 10 was set in the holding groove 21 of the board 20, and the inside of the tube 10 was sufficiently washed with distilled water, and then, filled with distilled water. Next, the washed collecting container 30 was placed on the distal side of the tube 10, and 100 mL of distilled water was injected from the proximal side of the tube 10 using the injection tool 40 (syringe). Next, 100 mL of distilled water collected in the collecting container 30 was set in a particle counter (HIAC 9703, available from Beckman Coulter, Inc.) and particles were measured under the test conditions (amount to be collected per measurement: 10 mL, frequency of collection: 4 times, calculation method: discard the first collected liquid and use data of the liquid collected in the remaining three measurements to obtain the average value, unit: particles/10 mL). The measured value was recorded as a blank.

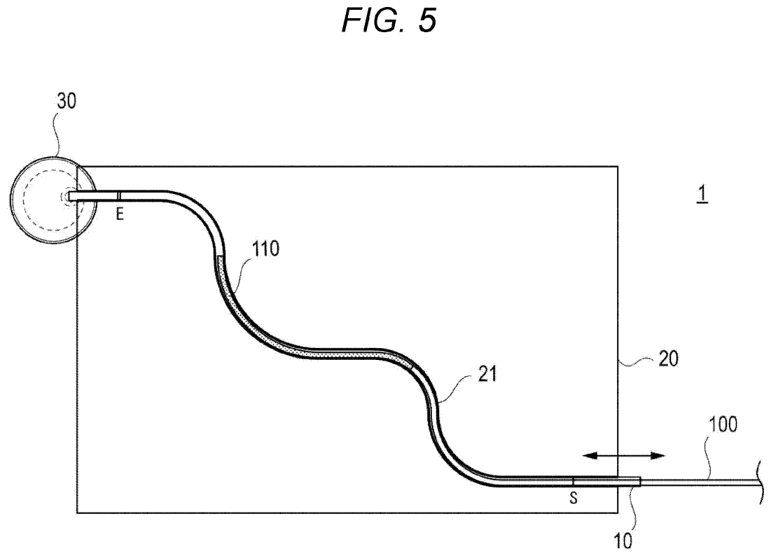

Subsequently, the lumen and the outer surface of each sample were washed with distilled water, and the distal end of each sample having the lumen filled with distilled water was placed at the insertion start point S of the tube 10. Next, as illustrated in FIG. 5, the reciprocating operation was performed 5 times by inserting the distal end of each sample from the insertion start point S to the insertion end point E and by subsequently retracting the distal end to the insertion start point S. In this reciprocating operation, when the distal end of each sample reaches the insertion end point E, the area provided with the lubricating layer 110 (area of 30 cm from the distal end) falls within the range between the insertion start point S and the insertion end point E.

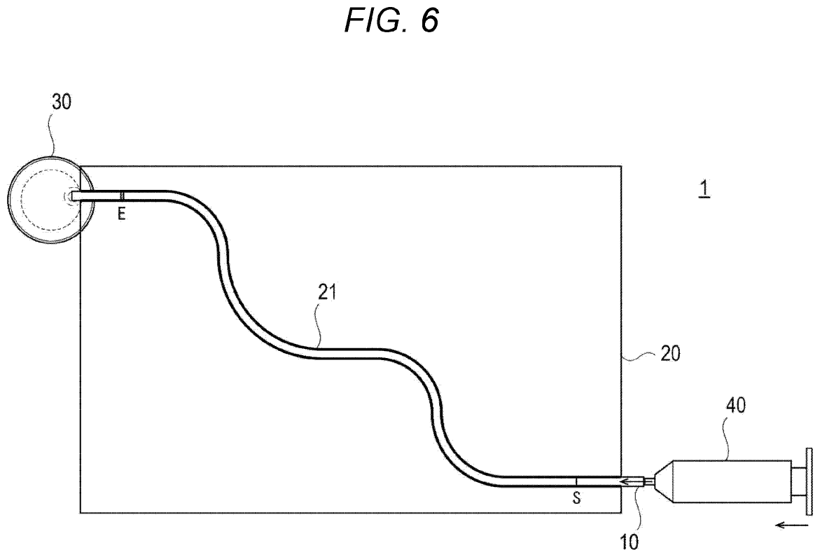

Subsequently, as illustrated in FIG. 6, 10 mL of distilled water was injected in a state where the distal end of each sample was placed at the insertion start point S. Next, each sample was removed from the tube 10, and 90 mL of distilled water was injected from the proximal side of the tube 10 to adjust the total amount of the collected liquid in the collecting container 30 ml to 100 mL.

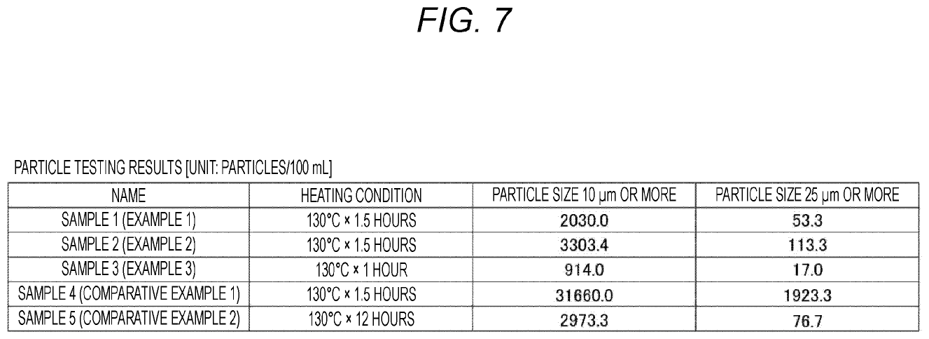

Next, the collected liquid in the collecting container 30 was set in the particle counter (HIAC 9703, available from Beckman Coulter, Inc.) and particles were measured under the test conditions (amount to be collected per measurement: 10 mL, frequency of collection: 4 times, calculation method: discard the first collected liquid and use data of the liquid collected in the remaining three measurements to obtain the average value, unit: particles/10 mL). The recorded blank value was subtracted from the measured value, the obtained value was multiplied by 10 and converted into the number of particles per 100 mL, and the converted value was recorded as a sum of particles. FIG. 7 shows measurement results of each sample.

### 4. Results

With regard to evaluation criteria of the testing method, the number of particles having a particle size of 10 μm or more was set to 5000 or less and/or the number of particles having a particle size of 25 μm or more was set to 200 or less. These criteria are indices of good peel resistance and a reduced frictional resistance of the lubricating layer 110.

As illustrated in FIG. 7, in each of Example 1 to Example 3, the number of particles having a particle size of 10 μm or more was 5000 or less, and the number of particles having a particle size of 25 μm or more was 200 or less. Particularly, in Example 3, the number of particles having a particle size of 10 μm or more was 914 and the number of particles having a particle size of 25 μm or more was 17, showing the smallest number and indicating excellent peel resistance. Therefore, Example 1 and Example 2 both exhibit excellent peel resistance, indicating that it is possible to reduce generation of particles derived from the lubricating layer 110 during a procedure. Furthermore, the time for heat drying the lubricating layers 110 in Example 1 and Example 2 was as short as 1.5 hours, and in Example 3, the time was as short as 1 hour. For this reason, the lubricating layers 110 of Example 1 to Example 3 suppressed excessive crosslinking or polymerization, had a small crosslinking density, and reduced damage to a hydrophilic group, which diminished the restriction on movement. Accordingly, even when subjected to friction, the lubricating layers 110 of Example 1 to Example 3 maintained surface lubricity. In other words, the lubricating layers 110 had a small or decreased frictional resistance.

Comparatively, in Comparative Example 1 and Comparative Example 2, a conventional lubricating layer composed of a lubricating resin was formed, and the number of particles having a particle size of 10 μm or more and the number of particles having a particle size of 25 μm or more both substantially exceeded the criteria. Since a non-lubricating resin was not added to the lubricating material in the lubricating layer 110 of Comparative Example 1, the peel resistance was relatively low, and the number of particles was nearly ten times more than the values obtained in Example 1 and Example 2. This result shows that adding a non-lubricating resin to the lubricating material is an important factor for enhancing the peel resistance of the lubricating layer 110 while maintaining the surface lubricity.

In Comparative Example 2, the number of particles was comparable with the values obtained in Example 1 and Example 2, but the heating time was 12 hours, which was 8 times longer than the time required in Example 1 and Example 2. This result shows that the fixing strength with respect to the elongated medical body 100 was enhanced in Comparative Example 2 due to long-duration heat drying. However, Comparative Example 2 deteriorated in surface lubricity due to the long-duration heat drying.

The detailed description above describes embodiments of a testing device for conducting a test of a lubricating layer formed on an elongated medical body which is inserted into and retracted from a body lumen, a testing method using the testing device, and an elongated medical body including a lubricating layer that has the number of particles equal to or less than a predetermined number when measured by the testing method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A testing device for measuring a number of particles generated from a lubricating layer formed on an elongated medical body, the testing device comprising:
a flexible tube provided with a lumen, the lumen extending from a distal end of the tube to a proximal end of the tube;
a board, the board including a holding groove having a recessed shape configured to hold the tube in a deformed state;
a collecting container configured to be placed on a distal side of the tube arranged in the holding groove and configured to store liquid flowing in from an injection tool; and
wherein the holding groove has a groove shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body, and the holding groove deforms and holds the tube according to the groove shape when the tube is fitted into the holding groove.

2. The testing device according to claim 1, further comprising:
the elongated medical body; and
wherein an insertion start point of the elongated medical body is set in a portion corresponding to the first straight portion of the holding groove, and an insertion end point of the elongated medical body is set in a portion corresponding to the third straight portion of the holding groove.

3. The testing device according to claim 2, wherein the elongated body is configured to be inserted into the tube and a portion of the elongated medical body provided with the lubricating layer is configured to be inserted and retracted in a reciprocating manner between the insertion start point and the insertion end point.

4. The testing device according to claim 2, wherein the insertion start point and the insertion end point of the elongated medical body are set at positions that secure at least a length equal to or longer than a predetermined length of the elongated medical body provided with the lubricating layer.

5. The testing device according to claim 4, wherein when the elongated medical body is inserted from the insertion start point to the insertion end point of the tube, the predetermined length provided with the lubricating layer is inserted into the tube.

6. The testing device according to claim 2, wherein the lubricating layer of the elongated medical body is at least 30 cm from a distal end of the elongated body, and a length from the insertion start point to the insertion end point is at least 30 cm or greater.

7. The testing device according to claim 1, wherein
the tube has an inside diameter of 4 mm, an outside diameter of 6 mm, and a total length of 1 m;
the board 20 has a length of 230 mm, a width of 380 mm, and a thickness of 19 mm; and
the holding groove has a groove depth of 6.2 mm, a groove width of 6.2 mm, the first straight portion has a length of 80 mm, the second straight portion has a length of 40 mm, the third straight portion has a length of 60 mm, the first meandering portion has a curved portion on the proximal side with a radius of curvature of 50 mm and a curved portion on the distal side with a radius of curvature of 40 mm, and the second meandering portion has a curved portion on the proximal side with a radius of curvature of 70 mm and a curved portion on the distal side with a radius of curvature of 40 mm.

8. A testing method using the testing device according to claim 1, the testing method comprising:
preparing the testing device and the elongated medical body provided with the lubricating layer in an area having a predetermined length;
setting a distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point;
retracting the elongated medical body from the tube and injecting the liquid into the tube in such a manner that collected liquid stored in the collecting container becomes 100 mL in total; and
using the collected liquid to measure the number of particles in the liquid and multiplying an obtained value by 10 to convert the obtained value into the number per 100 mL and regarding a converted value as a sum of the particles.

9. A testing method using the testing device of claim 1, the testing method comprising:
preparing the testing device and the elongated medical body provided with a lubricating layer having a predetermined length;
setting a distal end of the elongated medical body at an insertion start point of the tube, and repeating insertion and retraction of the elongated medical body a plurality of times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point;
retracting the elongated medical body from the tube and injecting a liquid into the tube such a manner that a predetermined amount of collected liquid is collected in the collecting container; and
measuring a number of particles in the predetermined amount of collected liquid.

10. The method according to claim 9, wherein the plurality of repeating the insertion and the retraction of the elongated medical body is 5 times.

11. The method according to claim 10, wherein
the predetermined amount of the collected liquid is 100 mL; and
multiplying an obtained value of the predetermined amount of the collected liquid by 10 to convert the obtained value into a number per 100 mL and regarding a converted value as a sum of the particles.

12. The method according to claim 9, further comprising:
placing the collecting container on a distal side of the tube and collecting the predetermined amount of the collected liquid in the collecting container.

13. The method according to claim 9, further comprising:
forming the lubricating layer by adding a non-lubricating material to a lubricating material.

14. The method according to claim 13, wherein the lubricating material is a hydrophilic material, and the non-lubricating material being one of vinyl chloride resin, urethane resin, and methylene diphenyl 4,4′-diisocyanate.

15. An elongated medical body comprising:
a main body; and
a lubricating layer formed on an outer surface of the main body in a predetermined area, the predetermined area having a distal end and a proximal end,
wherein the lubricating layer has no more than 5000 particles having a particle size of 10 μm or more and/or no more than 200 particles having a particle size of 25 μm or more,
when measured with a testing device that includes a tube having flexibility and provided with a lumen communicating a distal end and a proximal end of the tube and a collecting container placed on distal side of the tube and used to store liquid flowing in from an injection tool, the tube being deformed and held to have a shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body,
by a testing method comprising:
preparing the elongated medical body provided with the lubricating layer in an area having a predetermined length from the distal end toward the proximal end of the tube;
setting a distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point;
retracting the elongated medical body from the tube and injecting the liquid into the tube in such a manner that collected liquid stored in the collecting container becomes 100 mL in total; and
using the collected liquid to measure the number of particles in the liquid and multiplying an obtained value by 10 to convert the obtained value into the number per 100 mL and regarding a converted value as a sum of the particles.

16. The elongated medical body according to claim 15,
wherein the lubricating layer is formed by adding a non-lubricating material to a lubricating material;
the lubricating material being a hydrophilic material; and
the non-lubricating material being any of vinyl chloride resin, urethane resin, and methylene diphenyl 4,4′-diisocyanate.

17. The elongated medical body according to claim 15,
wherein the lubricating layer is formed on the outer surface of the main body in an area of 30 cm from the distal end of the elongated medical body; and
the main body has an outside diameter of 2.4 mm.

18. An elongated medical body comprising:
a main body; and
a lubricating layer formed on an outer surface of the main body in a predetermined area, the predetermined area having a distal end and a proximal end,
wherein the lubricating layer has no more than 5000 particles having a particle size of 10 μm or more and/or no more than 200 particles having a particle size of 25 μm or more,
when measured with a testing device that includes a tube having flexibility and provided with a lumen communicating a distal end and a proximal end of the tube, a board provided with a holding groove for holding the tube in a deformed state, and a collecting container placed on distal side of the tube and used to store liquid flowing in from an injection tool, the holding groove having a groove shape including a first straight portion, a first meandering portion adjacent to a distal end of the first straight portion, a second straight portion adjacent to a distal end of the first meandering portion, a second meandering portion adjacent to a distal end of the second straight portion, and a third straight portion adjacent to a distal end of the second meandering portion as seen from an insertion direction of the elongated medical body, and the tube being deformed and held according to the groove shape when fitted into the holding groove,
by a testing method comprising:
preparing the testing device and the elongated medical body provided with the lubricating layer in an area having a predetermined length from the distal end toward the proximal end of the tube;
setting a distal end of the elongated medical body at an insertion start point of the tube and repeating insertion and retraction 5 times by inserting the distal end of the elongated medical body from the insertion start point to an insertion end point of the tube in such a manner that a portion of the elongated medical body provided with the lubricating layer is completely inserted and by subsequently retracting the distal end of the elongated medical body to the insertion start point;
retracting the elongated medical body from the tube and injecting the liquid into the tube in such a manner that collected liquid stored in the collecting container becomes 100 mL in total; and
using the collected liquid to measure the number of particles in the liquid and multiplying an obtained value by 10 to convert the obtained value into the number per 100 mL and regarding a converted value as a sum of the particles.

19. The elongated medical body according to claim 18, wherein
the lubricating layer is formed by adding a non-lubricating material to a lubricating material;
the lubricating material being a hydrophilic material; and
the non-lubricating material being any of vinyl chloride resin, urethane resin, and methylene diphenyl 4,4′-diisocyanate.

20. The elongated medical body according to claim 18, wherein
the lubricating layer is formed on the outer surface of the main body in an area of 30 cm from the distal end of the elongated medical body; and
the main body has an outside diameter of 2.4 mm.
